(19) United States
(12) Patent Application Publication
(10) Pub. No.: US 2024/0139436 A1
(43) Pub. Date: May 2, 2024

(54) APPARATUS AND METHOD FOR CONTROLLING INJECTION OF DRUG INTO OBJECT, AND RECORDING MEDIUM STORING COMPUTER PROGRAM FOR PERFORMING THE METHOD

(71) Applicants: SEOUL NATIONAL UNIVERSITY R&DB FOUNDATION, Seoul (KR); UIF (UNIVERSITY INDUSTRY FOUNDATION), YONSEI UNIVERSITY, Seoul (KR); IUCF-HYU (Industry-University Cooperation Foundation Hanyang University), Seoul (KR)

(72) Inventors: Young Bin Choy, Seoul (KR); Han Bi Ji, Seoul (KR); Chang Hee Min, Seoul (KR); Jae Hoon Han, Seoul (KR); Won Seok Chang, Seoul (KR); Jun Won Park, Seoul (KR); Chang-Hwan Im, Seoul (KR); Kyeong-Gu Lee, Seoul (KR)

(73) Assignees: SEOUL NATIONAL UNIVERSITY R&DB FOUNDATION, Seoul (KR); UIF (UNIVERSITY INDUSTRY FOUNDATION), YONSEI UNIVERSITY, Seoul (KR); IUCF-HYU (Industry-University Cooperation Foundation Hanyang University), Seoul (KR)

(21) Appl. No.: 18/497,431

(22) Filed: Oct. 30, 2023

(30) Foreign Application Priority Data
Oct. 28, 2022 (KR) 10-2022-0141618

(51) Int. Cl. A61M 5/42

(52) U.S. Cl. CPC A61M 5/427; A61M 2205/502; A61M 2205/52

(57) ABSTRACT

A method of controlling injection of a drug includes receiving a captured medical image of an object, identifying an object of interest from the medical image, based on a position and a shape of the object of interest, determining a drug injection position, based on the position and the shape of the object of interest, determining a plurality of electrode insertion positions, the plurality of electrodes being configured to supply a current to move the drug, defining a drug movement path based on a shape of the identified object of interest, generating current sequence information to move the drug along the drug movement path, the current sequence information defining a current applied over time to each of the plurality of electrodes, and outputting the current sequence information to a current source apparatus.

### CROSS-REFERENCE TO RELATED APPLICATION

This application is based on and claims priority under 35 U.S.C. § 119 to Korean Patent Application No. 10-2022-0141618, filed on Oct. 28, 2022, in the Korean Intellectual Property Office, the disclosure of which is incorporated by reference herein in its entirety.

### BACKGROUND

### 1. Field

Embodiments relate to an apparatus for controlling injection of a drug, a method of controlling injection of a drug, and a computer-readable recording medium having recorded thereon a program for executing, on a computer, the method of controlling injection of a drug.

### 2. Description of the Related Art

Various methods are used to introduce drugs into lesions in an object. When surgical resection of a lesion in an object is difficult, treatment using drugs may be used. However, when it is difficult to approach an area around the lesion in the object, it is difficult to selectively deliver a drug to a position where the lesion occurred. In addition, when a lesion is positioned near a sensitive normal tissue, it is difficult to selectively apply a drug only to the lesion. For example, brain tumors are difficult to be completely resected via a single surgical resection, require additional surgery and follow-up treatment, and often have a high recurrence rate. In this case, drug treatment for a brain tumor may be performed, but it is difficult to selectively apply a drug to the brain tumor inside the brain.

When the drug fails to be selectively applied to the lesion, the rate at which the drug is delivered to the lesion decreases, and accordingly, it is difficult to obtain a drug therapeutic effect. Moreover, when the drug fails to be selectively applied to the lesion, the drug may be delivered to a normal tissue, which may cause side effects of damaging the normal tissue.

### SUMMARY

Embodiments provide an apparatus and method for moving and distributing a drug to a region of interest in an object by precisely controlling a current in the object, and a recording medium storing a program for performing the method.

Additional aspects will be set forth in part in the description which follows and, in part, will be apparent from the description, or may be learned by practice of the presented embodiments of the disclosure.

According to an aspect of an embodiment, there is provided a method of controlling injection of a drug, the method including receiving a captured medical image of an object, identifying an object of interest from the medical image, based on a position and a shape of the object of interest, determining a drug injection position where a drug is to be injected, based on the position and the shape of the object of interest, determining a plurality of electrode insertion positions where a plurality of electrodes are to be inserted, respectively, the plurality of electrodes being configured to supply a current to move the drug, defining a drug movement path based on a shape of the identified object of interest, generating current sequence information to move the drug along the drug movement path, the current sequence information defining a current to be applied over time to each of the plurality of electrodes, and outputting the current sequence information to a current source apparatus, the current source apparatus being configured to supply the current to the plurality of electrodes.

In addition, according to an embodiment, the defining of the drug movement path may include defining the drug movement path to move the drug along a surface of the object of interest.

In addition, according to an embodiment, the object may be a brain of a patient or an animal, and a magnitude of the current supplied to the plurality of electrodes may be defined as a 2 mA or less.

In addition, according to an embodiment, the current sequence information may include at least one of a current value of the current applied to each of the plurality of electrodes, a duty cycle, a total current application duration, and an on/off timing or a combination thereof.

In addition, according to an embodiment, the generating of the current sequence information may include determining the total current application duration based on a movement distance for which the drug moves along the drug movement path and the duty cycle.

In addition, according to an embodiment, the generating of the current sequence information may further include determining an on/off timing of each of the plurality of electrodes to move the drug along the drug movement path.

In addition, according to an embodiment, the generating of the current sequence information may further include determining a movement speed of the drug based on the shape of the object of interest, and determining the duty cycle based on the movement speed of the drug.

In addition, according to an embodiment, the determining of the movement speed of the drug may include increasing the movement speed of the drug as electrical conductivity of the object of interest is lower.

In addition, according to an embodiment, the injection of the drug may be performed by inserting a drug injection apparatus at the drug injection position in the object and injecting the drug from the drug injection apparatus into the object, and when the drug injection apparatus includes an electrode, the drug injection position may correspond to one of the plurality of electrode insertion positions.

In addition, according to an embodiment, the object of interest may correspond to a lesion or a tumor in the object.

In addition, according to an embodiment, the medical image may include at least one of a magnetic resonance imaging (MRI) image, a computed tomography (CT) image, an X-ray image, or an ultrasound image.

In addition, according to an embodiment, the drug may include at least one of polar molecules, ions, or particles.

In addition, according to an embodiment, the drug may include at least one of a tissue-specific adhesion functional group for the object of interest, a contrast agent, or a therapeutic agent.

In addition, according to an embodiment, the method of controlling injection of a drug may further include displaying, on the medical image, object-of-interest region information corresponding to the object of interest, information about the drug injection position, information about the plurality of electrode insertion positions, and information about the drug movement path.

According to another aspect of an embodiment, there is provided an apparatus for controlling injection of a drug, the apparatus including an input interface, an output interface, a memory storing at least one instruction, and at least one processor connected to the memory, wherein the at least one processor is configured to execute the at least one instruction to receive a captured medical image of an object through the input interface, identify an object of interest from the medical image, based on a position and a shape of the object of interest, determine a drug injection position where a drug is to be injected, based on the position and the shape of the object of interest, determine a plurality of electrode insertion positions where a plurality of electrodes are to be inserted, the plurality of electrodes being configured to supply a current to move the drug, define a drug movement path defined based on a shape of the identified object of interest, generate current sequence information to move the drug along the drug movement path, the current sequence information defining a current to be applied over time to each of the plurality of electrodes, and output the current sequence information to a current source apparatus through the output interface, the current source apparatus being configured to supply the current to the plurality of electrodes.

According to another aspect of an embodiment, there is provided a computer-readable recording medium having recorded thereon a program for executing, on a computer, the method of controlling injection of a drug.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram of a drug injection control system according to an embodiment;

FIG. 2 is a diagram of a drug injection control apparatus according to an embodiment;

FIG. 3 is a flowchart of a drug injection control method according to an embodiment;

FIG. 4 is a diagram illustrating a process of identifying an object of interest in a medical image, according to an embodiment;

FIG. 5 is a diagram illustrating a drug injection position and a plurality of electrode insertion positions on a medical image, according to an embodiment;

FIG. 6 is a diagram illustrating an arrangement of an object of interest and electrodes, according to an embodiment;

FIG. 7 is a diagram illustrating an arrangement of an object of interest and electrodes, according to an embodiment;

FIG. 8 is a diagram of a drug injection apparatus according to an embodiment;

FIG. 9 is a diagram illustrating a drug injection position and a plurality of electrode insertion positions on a medical image, according to an embodiment;

FIG. 10 is a diagram illustrating a process of setting a drug movement path, according to an embodiment;

FIG. 11 is a diagram illustrating a process of deriving a current application condition, according to an embodiment;

FIG. 12 is a diagram illustrating an example of a current field distribution calculated based on anatomical characteristics of an object, according to an embodiment;

FIG. 13 is a diagram illustrating a state in which a drug is distributed by diffusion and a state in which the drug is distributed over time in a current field, according to an embodiment;

FIG. 14 is a diagram illustrating a state in which a drug is moved by controlling each electrode, according to an embodiment;

FIG. 15 is an experimental example of moving a drug by applying a current to an electrode, according to an embodiment;

FIG. 16 is an experimental example illustrating a state in which a drug moves in a matrix according to a comparative example;

FIG. 17 is a diagram of a configuration for adjusting a movement rate of a drug, according to an embodiment;

FIG. 18 is a diagram of a structure of a drug according to an embodiment;

FIG. 19 is a diagram illustrating current sequence information according to an embodiment;

FIG. 20 is a diagram illustrating a process of identifying an object of interest, according to an embodiment;

FIG. 21 is a diagram illustrating a process of setting a drug movement path, according to an embodiment;

FIG. 22 is a diagram illustrating a state in which a drug is moved by applying a current to four electrodes, according to an embodiment;

FIG. 23 is a diagram illustrating an arrangement of electrodes and a current application process, according to an embodiment; and

FIG. 24 is a diagram of a drug injection control system according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

This specification clarifies the scope of the claims, and the principles of embodiments are described so that one of ordinary skill in the art to which the embodiments of this disclosure belong can practice the embodiments recited in the claims. The disclosed embodiments may be implemented in various forms.

Throughout the specification, the same elements are denoted by the same reference numerals. This specification does not cover all elements of the embodiments, and descriptions that are common in the art to which the embodiments of the disclosure belong or that are redundant between the embodiments are omitted. As used herein, the terms “module”, “ . . . or/er”, or “unit” may be implemented in one or more combinations of software, hardware, or firmware. In some embodiments, a plurality of “modules”, “ . . . ors/ers”, or “units” may be implemented as a single element, a single “module”, a “ . . . or/er”, or a “unit” that may include a plurality of elements.

While describing the embodiments, detailed descriptions of related well-known technology may be omitted when it is deemed that they may unnecessarily obscure the essence of the disclosure. In addition, numbers (e.g., first, second, etc.) used in the description the specification are merely used to identify symbols to distinguish one element from another.

Moreover, as used herein, when an element is referred to as being “connected” or “coupled” to another element, it may be directly connected or coupled to the other element, but it will be understood to include that the element is connected or coupled to the other element with another element therebetween, unless specified otherwise.

Various embodiments and the principles of operation of the embodiments will be described below with reference to the accompanying drawings.

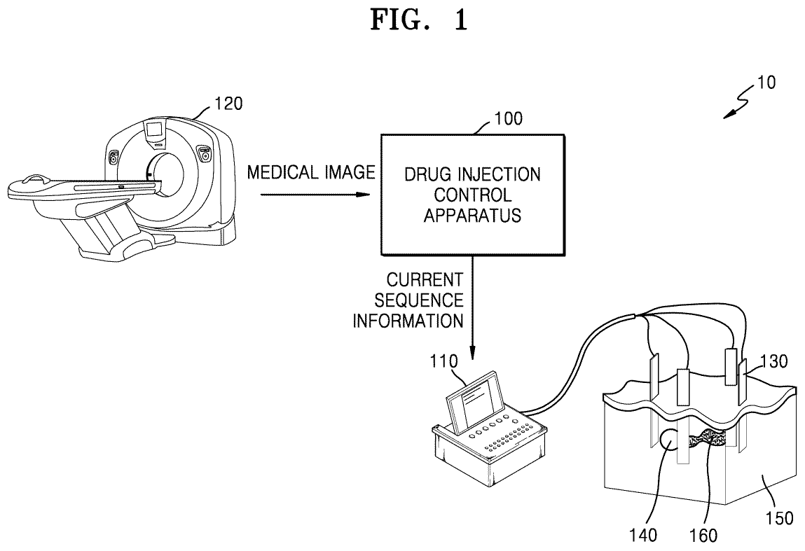

FIG. 1 is a diagram of a drug injection control system according to an embodiment.

According to an embodiment, a drug injection control system 10 injects a drug into an object of interest 160 in an object 150 by controlling a current in the object 150. The drug injection control system 10 includes a drug injection control apparatus 100, a current source apparatus 110, and a medical imaging apparatus 120.

The medical imaging apparatus 120 generates a medical image by photographing the object 150. The medical imaging apparatus 120 may correspond to, for example, a magnetic resonance imaging (MRI) apparatus, a computed tomography (CT) apparatus, an ultrasound imaging apparatus, or an X-ray imaging apparatus. The medical image may correspond to an MRI image, a CT image, an ultrasound image, or an X-ray image.

According to an embodiment, the medical image may correspond to a three-dimensional (3D) medical image. For example, the medical image may correspond to a medical image rendered in three dimensions.

According to an embodiment, the medical image may include a plurality of cross-sectional images. For example, the medical image may correspond to a plurality of medical images captured from a plurality of directions, such as a front, side, top, or rear view.

The drug injection control apparatus 100 may receive the medical image. According to an embodiment, the drug injection control apparatus 100 may directly receive the medical image from the medical imaging apparatus 120. According to another embodiment, the drug injection control apparatus 100 may receive a pre-captured and stored medical image from a certain external apparatus or server.

According to an embodiment, a plurality of electrodes 130 may be inserted into the object 150. The current source apparatus 110 may be connected to the plurality of electrodes 130 and output a current signal to the plurality of electrodes 130. The current source apparatus 110 may generate the current signal based on current sequence information input from the drug injection control apparatus 100 and may output the current signal. The current sequence information defines a current to be applied over time to each of the plurality of electrodes 130. The current sequence information defines current values, a duty cycle, a total current application duration, and an on/off timing of the plurality of electrodes 130. The current output from the current source apparatus 110 may be applied to the plurality of electrodes 130 to generate a current in the object 150. A drug 140 may be moved within the object 150 by the current.

The drug 140 may correspond to a material or ions having polarity. The drug 140 may include at least one of polar molecules, ions, or particles. The drug 140 having polarity may be moved by the current. The drug 140 may be moved according to the electrophoretic principle or iontophoresis principle.

The drug 140 may correspond to molecules, ink, or the like. According to an embodiment, the drug 140 may correspond to ink in the form of small molecules. Also, the drug 140 may correspond to a theranostic ink in the form of small molecules in which a contrast agent, a therapeutic agent, and a targeting moiety are conjugated.

The object 150 may include a person, an animal, or a part thereof. For example, the object 150 may include a body part (such as an organ) or a phantom. The object 150 may correspond to, for example, the brain of a person or an animal. Also, the object 150 may correspond to a patient subject to a treatment or examination.

The drug injection control apparatus 100 may determine, based on the medical image, a drug injection position where a drug is to be injected into the object 150 and a plurality of electrode insertion positions where a plurality of electrodes are to be inserted. Also, the drug injection control apparatus 100 may define a drug movement path based on the medical image. In addition, the drug injection control apparatus 100 may generate the current sequence information to move the drug 140 along the drug movement path. The drug injection control apparatus 100 may output the current sequence information to the current source apparatus 110.

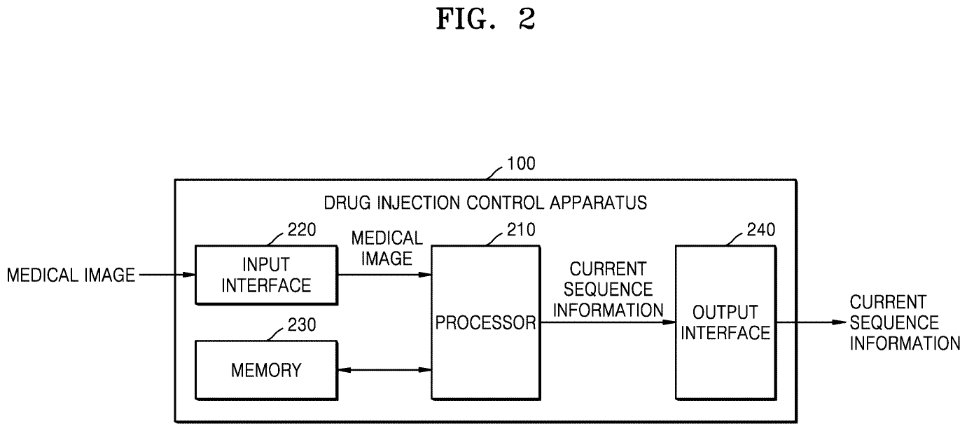

FIG. 2 is a diagram of a drug injection control apparatus according to an embodiment.

According to an embodiment, the drug injection control apparatus 100 may be implemented as various types of electronic apparatuses. The drug injection control apparatus 100 may be implemented in the form of, for example, a workstation, a personal computer (PC), a laptop computer, a mobile apparatus, a tablet PC, or the like.

The drug injection control apparatus 100 includes a processor 210, an input interface 220, a memory 230, and an output interface 240.

The processor 210 controls an overall operation of the drug injection control apparatus 100. The processor 210 may be implemented as one or more processors. The processor 210 may execute an instruction or command stored in the memory 230 to perform a certain operation. Also, the processor 210 controls operations of components provided in the drug injection control apparatus 100. The processor 210 may include at least one of a central processing unit (CPU), a graphics processing unit (GPU), a microprocessor, or a neural processing unit (NPU) or a combination thereof.

The input interface 220 receives a medical image from an external apparatus. The input interface 220 may correspond to, for example, a communication module that communicates with the external apparatus, an input terminal, or the like. According to an embodiment, the input interface 220 receives the medical image from the medical imaging apparatus 120. As another example, the input interface 220 receives the medical image from the external apparatus such as a user apparatus or a server. The input interface 220 may receive the medical image from various electronic apparatuses or combinations of various electronic apparatuses.

Also, the input interface 220 may include an apparatus that receives a user input. The input interface 220 may include, for example, a keyboard, a keypad, a mouse, a touch pad, a touch screen, a button, a key, a dial, a wheel mouse, or a jog dial.

The memory 230 stores various types of information, data, instructions, programs, etc. required for the operation of the drug injection control apparatus 100. The memory 230 may include at least one of a volatile memory or a nonvolatile memory or a combination thereof. The memory 230 may include at least one type of storage medium among a flash memory-type memory, a hard disk-type memory, a multimedia card micro-type memory, a card-type memory (for example, secure digital (SD) or extreme digital (XD) memory), random access memory (RAM), static RAM (SRAM), read-only memory (ROM), electrically erasable-programmable ROM (EEPROM), programmable ROM (PROM), a magnetic memory, a magnetic disk, and an optical disk. Also, the memory 230 may correspond to a web storage or cloud server that performs a storage function over the Internet.

The output interface 240 outputs current sequence information to the external apparatus. The output interface 240 may be connected to the current source apparatus 110 and output the current sequence information to the current source apparatus 110. The output interface 240 may correspond to, for example, a communication module, an output terminal, or the like.

Also, the output interface 240 may include a user interface apparatus that outputs information and data regarding the operation of the drug injection control apparatus 100. The output interface 240 may include, for example, a display, a speaker, a touch screen, or a printer.

The processor 210 may store the medical image received through the input interface 220 in the memory 230. The processor 210 generates the current sequence information based on the medical image stored in the memory 230.

The processor 210 identifies an object of interest 160 from the medical image. The object of interest 160 is an object to which the drug 140 is to be delivered by the drug injection control apparatus 100. The object of interest 160 may correspond to, for example, a lesion, a tumor, an abnormal tissue, an infected tissue, a tissue to be removed, a tissue to be examined, a tissue to be treated, or the like.

According to an embodiment, the processor 210 may recognize and identify the object of interest 160 by using a certain algorithm. According to another embodiment, the processor 210 may receive a user input for setting the object of interest 160 through the input interface 220 and identify the object of interest 160 based on the user input. A method of identifying the object of interest 160 may be implemented as one or an automatic identification method and a user input-based identification method, or both of the methods together. When the object of interest 160 is identified, the processor 210 may provide information about the object of interest 160 on the medical image by using the output interface 240. For example, the processor 210 may provide the information about the object of interest 160 by displaying an identifier indicating an edge of the object of interest 160 on the medical image.

When the object of interest 160 is identified, the processor 210 determines a drug injection position based on a position and shape of the object of interest 160. The processor 210 determines the drug injection position to inject a drug into the vicinity of the object of interest 160 based on the position of the object of interest 160 to move the drug to the object of interest 160. Also, the processor 210 may determine a starting point to move the drug in the vicinity of the object of interest 160 based on the shape of the object of interest 160, and determine a position in the vicinity of the determined starting point as the drug injection position. The processor 210 may automatically determine the drug injection position by a certain algorithm or may determine the drug injection position based on a user input. When the drug injection position is determined, the processor 210 may provide information about the drug injection position on the medical image through the output interface 240. When the drug injection position is automatically determined, the processor 210 may adjust the drug injection position based on the user input. Also, when the drug injection position is automatically determined, the processor 210 may finalize the drug injection position when a user approves the determined drug injection position.

According to an embodiment, when the drug injection position is automatically determined, the processor 210 determines, as the drug injection position, a position where a drug injection apparatus is accessible in a surrounding area within a reference distance from the object of interest 160. The processor 210 may determine the drug injection position in an area of the surrounding area excluding an access restricted area where access of the drug injection apparatus is restricted.

When the drug injection position is determined, the processor 210 determines electrode insertion positions where the plurality of electrodes 130 are to be inserted, based on the position and shape of the object of interest 160. The processor 210 determines positions where the plurality of electrodes 130 are to be inserted, respectively, in the vicinity of the object of interest 160, based on the position of the object of interest 160. Also, the processor 210 determines the positions where the plurality of electrodes 130 are to be inserted, respectively, based on the shape of the object of interest 160. The processor 210 determines a position where each of the plurality of electrodes 130 may be inserted and arranged in the object 150, based on a shape and size of each of the plurality of electrodes 130. The processor 210 defines an area of a surrounding area of the object of interest 160, where each of the plurality of electrodes 130 is accessible. The processor 210 determines the electrode insertion positions except for an area where a tissue with a risk of damage is present in the object 150. Also, the processor 210 determines the electrode insertion positions in an area within a reference distance from the object of interest 160.

The plurality of electrodes 130 control the movement of the drug 140 by generating a current field in a 3D space. The processor 210 determines the electrode insertion positions of the plurality of electrodes 130, such that the plurality of electrodes 130 generate the current field in the 3D space to control the movement of the drug 140. The plurality of electrodes 130 generate a current field in the object 150 by applying a current to the object 150. The drug 140 may correspond to a polar material or ions and may be moved by the current field. The drug injection control apparatus 100 controls a movement direction and speed of the drug 140 by controlling a direction and magnitude of the current field generated by the plurality of electrodes 130.

The plurality of electrodes 130 may correspond to insertable electrodes in the form of catheters or plates. The electrodes 130 may each include a material such as stainless steel, platinum, or silicon. The electrodes 130 may each have a size of 1 mm or greater in diameter or width. As another example, the plurality of electrodes 130 may correspond to patch-type electrodes attached to the skin of the object 150. As another example, the plurality of electrodes 130 may include a combination of insertable electrodes and patch-type electrodes.

According to an embodiment, the plurality of electrodes 130 include two electrodes. The processor 210 determines the electrode insertion positions such that two electrodes 130 are respectively inserted with the object of interest 160 and the drug injection position therebetween.

According to another embodiment, the plurality of electrodes 130 include four electrodes. Four electrodes 130 are arranged to generate current fields in different axes with the object of interest 160 therebetween. For example, the two electrodes 130 are arranged with the object of interest 160 therebetween in a first-axis direction. The other two electrodes 130 are arranged with the object of interest 160 therebetween in a second-axis direction different from the first-axis direction.

When the plurality of electrode insertion positions are determined, the processor 210 defines a drug movement path based on the shape of the identified object of interest 160. The drug injection control apparatus 100 may move the drug 140 to the vicinity of the object of interest 160 to deliver the drug 140 to the object of interest 160. For example, the drug movement path is set such that the drug 140 is moved along the surface of the object of interest 160. For example, the drug movement path may be set to apply the drug 140 to the surface of the object of interest 160. As another example, the drug movement path may be set to pass through the surrounding area of the object of interest 160 to increase the concentration of the drug 140 in the surrounding area of the object of interest 160.

When the drug movement path is defined, the processor 210 generates current sequence information that defines a current to be applied over time to each of the plurality of electrodes 130 to move the drug 140 along the drug movement path. The processor 210 controls the plurality of electrodes 130 to apply a current for generating a current field to the object 150 to move the drug 140 along the drug movement path. For example, the processor 210 generates a current field by applying a current to the plurality of electrodes 130 arranged on both sides of the object of interest 160 in the first-axis direction to move the drug 140 in the first-axis direction. The processor 210 defines a current to be applied over time to each of the electrodes 130 to generate a current for moving the drug 140 along the drug movement path. As described above, the processor 210 generates the current sequence information that defines the current to be applied over time to each of the electrodes 130. The current sequence information may include at least one of a current value of a current applied to each of the plurality of electrodes 130, a duty cycle, a total current application duration, and an on/off timing or a combination thereof.

When the current sequence information is generated, the processor 210 outputs the current sequence information to the current source apparatus 110. The processor 210 outputs the current sequence information to the current source apparatus 110 through the output interface 240.

In addition, when the drug injection position is determined, the drug 140 is injected into the drug injection position within the object 150. Also, when the electrode insertion positions are determined, the plurality of electrodes 130 are inserted into the electrode insertion positions within the object 150. The injection of the drug 140 and the insertion of the electrodes 130 may be performed by a medical practitioner. As another example, the injection of the drug 140 and the insertion of the electrodes 130 may be performed by an apparatus such as a surgical robot.

The current source apparatus 110 receives the current sequence information. When the injection of the drug 140 and the insertion of the electrodes 130 are completed, the current source apparatus 110 generates a current to be applied to the plurality of electrodes 130 according to the current sequence information. Also, the current source apparatus 110 applies a current to each of the plurality of electrodes 130. The drug 140 is moved along the drug movement path within the object 150 according to the current applied to the plurality of electrodes 130.

Operation information about the current source apparatus 110 may be input to the drug injection control apparatus 100. For example, information about the current output from the current source apparatus 110 to each of the plurality of electrodes 130 may be fed back to the drug injection control apparatus 100 in real time. The drug injection control apparatus 100 may output current output information through the output interface 240 based on feedback information received from the current source apparatus 110. According to an embodiment, the drug injection control apparatus 100 may generate information about the movement of the drug 140 estimated according to the current output information and output the information through the output interface 240.

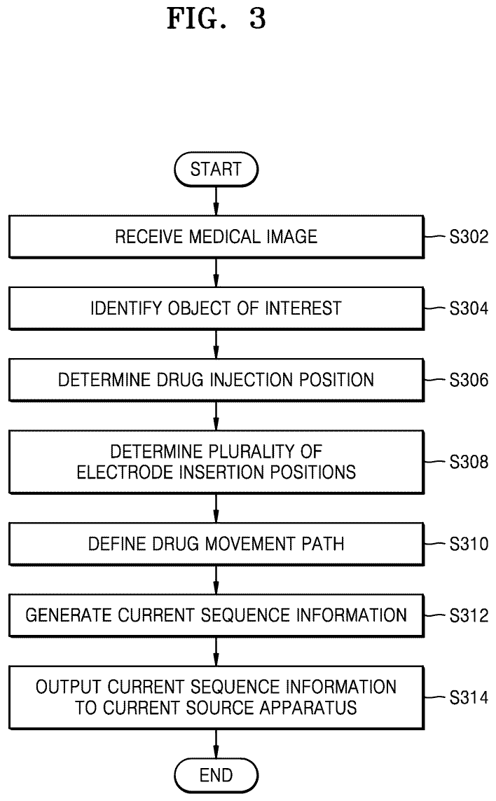

FIG. 3 is a flowchart of a drug injection control method according to an embodiment.

According to an embodiment, each operation of the drug injection control method may be performed by various types of electronic apparatuses each including an input interface, a processor, and an output interface. In the disclosure, an embodiment in which the drug injection control apparatus 100 according to embodiments performs the drug injection control method will be mainly described. Accordingly, the embodiments described for the drug injection control apparatus 100 are applicable to embodiments of the drug injection control method, and on the contrary, the embodiments that will be described for the drug injection control method are also applicable to the embodiments of the drug injection control apparatus 100. The drug injection control method according to the embodiments of the disclosure is not limited to being performed by the drug injection control apparatus 100 described in the disclosure and may be performed by various types of drug injection control apparatuses.

In operation S302, the drug injection control apparatus 100 receives a medical image from an external apparatus. The drug injection control apparatus 100 may receive the medical image from the medical imaging apparatus 120. The medical image may correspond to an MRI image, a CT image, an ultrasound image, or an X-ray image. Also, the medical image may correspond to a 3D medical image. As another example, the medical image may include a plurality of cross-sectional images captured in a plurality of directions.

Next, in operation S304, the drug injection control apparatus 100 identifies an object of interest 160 from the medical image. The drug injection control apparatus 100 may automatically identify the object of interest 160 by a certain algorithm or may identify the object of interest 160 based on a user input.

Next, in operation S306, the drug injection control apparatus 100 determines a drug injection position based on a position and shape of the object of interest 160. The drug injection control apparatus 100 may automatically determine the drug injection position by a certain algorithm or may determine the drug injection position based on a user input. The drug injection control apparatus 100 determines the drug injection position to inject a drug into the vicinity of the object of interest 160 to move the drug to the object of interest 160. The drug injection control apparatus 100 may automatically determine the drug injection position by the certain algorithm or may determine the drug injection position based on the user input.

Next, in operation S308, when the drug injection position is determined, the drug injection control apparatus 100 determines electrode insertion positions where a plurality of electrodes 130 are to be inserted, based on the position and shape of the object of interest 160. The drug injection control apparatus 100 determines a position where each of the plurality of electrodes 130 is to be inserted and arranged in the object 150, based on a shape and size of each of the plurality of electrodes 130. The drug injection control apparatus 100 defines an area of a surrounding area of the object of interest 160, where each of the plurality of electrodes 130 is accessible. The drug injection control apparatus 100 determines the electrode insertion positions except for an area where a tissue with a risk of damage is present in the object 150. Also, the drug injection control apparatus 100 determines the electrode insertion positions in an area within a reference distance from the object of interest 160. The plurality of electrodes 130 may be provided in various numbers. The plurality of electrodes 130 may include, for example, two electrodes or four electrodes.

Next, when a drug movement path is defined, in operation S310, the drug injection control apparatus 100 generates current sequence information that defines a current to be applied over time to each of the plurality of electrodes 130 to move the drug 140 along the drug movement path. The current sequence information may include at least one of a current value of a current applied to each of the plurality of electrodes 130, a duty cycle, a total current application duration, and an on/off timing or a combination thereof.

Next, when the current sequence information is generated, in operation S314, the drug injection control apparatus 100 outputs the current sequence information to the current source apparatus 110.

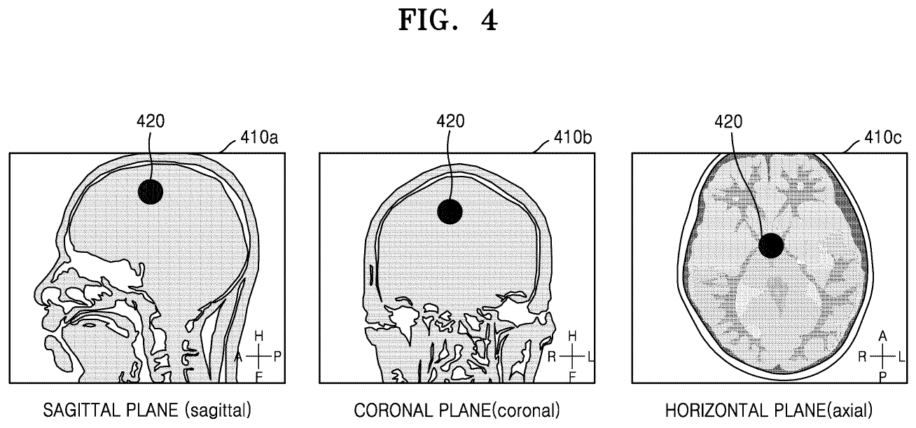

FIG. 4 is a diagram illustrating a process of identifying an object of interest in a medical image, according to an embodiment.

According to an embodiment, the drug injection control apparatus 100 identifies an object of interest 420 from medical images 410a, 410b, and 410c. The medical images 410a, 410b, and 410c may include cross-sectional medical images in a plurality of directions. For example, the medical images 410a, 410b, and 410c may include a sagittal medical image 410a, a coronal medical image 410b, and a horizontal (axial) medical image 410c. As another example, the medical images 410a, 410b, and 410c may correspond to 3D medical images. In the disclosure, an embodiment in which the medical images 410a, 410b, and 410c include a plurality of cross-sectional medical images 410a, 410b, and 410c will be mainly described. However, the embodiment is not limited to including the plurality of cross-sectional medical images 410a, 410b, and 410c.

The drug injection control apparatus 100 identifies the object of interest 420 from the plurality of medical images 410a, 410b, and 410c. According to an embodiment, the drug injection control apparatus 100 identifies the object of interest 420 from the plurality of medical images 410a, 410b, and 410c based on a user input.

According to an embodiment, a user may select the object of interest 420 by directly selecting an edge of the object of interest 420. The drug injection control apparatus 100 defines a boundary of the object of interest 420 based on the edge of the object of interest 420 input by the user.

According to another embodiment, the drug injection control apparatus 100 recognizes objects in the medical images 410a, 410b, and 410c by segmenting the medical images 410a, 410b, and 410c. The drug injection control apparatus 100 displays, on the output interface 240, information about the objects recognized in the medical images 410a, 410b, and 410c. The drug injection control apparatus 100 receives, through the input interface 220, a user input for selecting one of the recognized objects. The drug injection control apparatus 100 defines an object selected by the user as the object of interest 420.

A method of designating an edge of the object of interest 420 by a user and a method of receiving a user input for selecting one of objects recognized through a segmentation process as the object of interest 420 may be provided together.

According to an embodiment, the drug injection control apparatus 100 may display the medical images 410a, 410b, and 410c and information about the identified object of interest 420 through the output interface 240. The drug injection control apparatus 100 may display, on the medical images 410a, 410b, and 410c, an indicator indicating the edge of the object of interest 420.

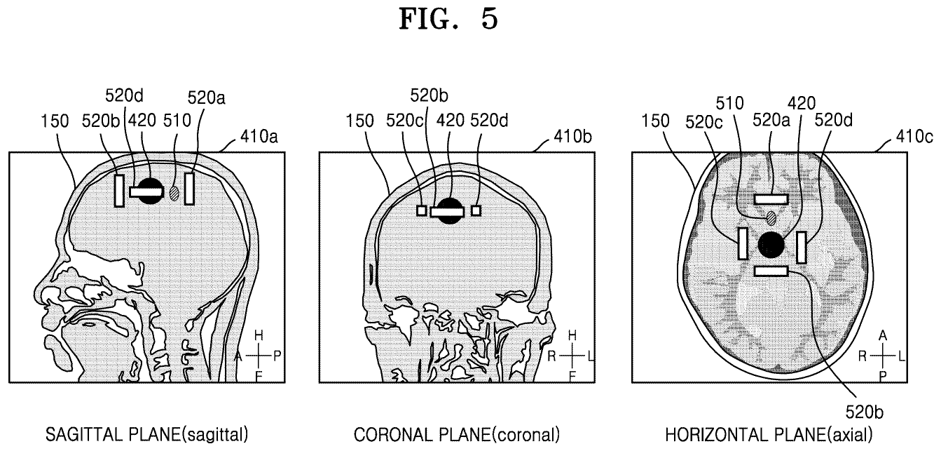

FIG. 5 is a diagram illustrating a drug injection position and a plurality of electrode insertion positions on a medical image, according to an embodiment.

According to an embodiment, the drug injection control apparatus 100 determines a drug injection position 510 and a plurality of electrode insertion positions 520a, 520b, 520c, and 520d within the object 150 from the medical images 410a, 410b, and 410c.

The drug injection control apparatus 100 determines the drug injection position 510 in a surrounding area of the object of interest 420. When a drug moves within the object 150, the drug injection control apparatus 100 determines a drug injection position in a surrounding area within a reference distance from the object of interest 420, such that the drug may easily move to the object of interest 420. For this, the drug injection control apparatus 100 defines a surrounding area within a reference distance from the object of interest 420 as a surrounding area.

Next, the drug injection control apparatus 100 determines, in the surrounding area, a position where the drug injection apparatus is insertable.

The drug is injected into the object 150 by the drug injection apparatus. The drug injection apparatus has a drug delivery structure and a drug discharge structure. The drug injection apparatus corresponds to, for example, a catheter, a needle, or the like.

When the drug injection apparatus is inserted into the object 150, the drug injection apparatus needs to be inserted not to damage a tissue of the object 150 while approaching the object of interest 420. However, the drug injection apparatus has a certain shape and size, and thus, the drug injection apparatus may damage the tissue of the object 150 or cause friction with the tissue of the object 150 when inserted into the object 150. According to an embodiment, when the drug injection apparatus is inserted into the object 150, the drug injection control apparatus 100 determines the drug injection position 510 where the drug injection apparatus is to be placed, in the surrounding area of the object of interest 420, such that the tissue of the object 150 is not damaged. The drug injection control apparatus 100 may determine, based on a shape of the object of interest 420, the drug injection position 510 such that the tissue of the object 150 or the object of interest 420 is not damaged by the drug injection apparatus.

According to an embodiment, the drug injection control apparatus 100 defines an access restricted area where access of the drug injection apparatus is restricted in the object 150. For example, the drug injection control apparatus 100 defines, as the access restricted area, an area where a tissue or organ with a risk of damage is present in the object 150. When the access restricted area is defined in the surrounding area, the drug injection control apparatus 100 determines a drug injection apparatus arrangement area where the drug injection apparatus may be arranged, among areas excluding the access restricted area. The drug injection control apparatus 100 may determine a drug injection apparatus arrangement area where the drug injection apparatus may be arranged, based on the size and shape of the drug injection apparatus. The drug injection control apparatus 100 may determine, as the drug injection apparatus arrangement area, an area that is close to the object of interest 160 and easily accessible to the electrodes 130, in an area where the drug injection apparatus may be arranged.

When the drug injection apparatus arrangement area where the drug injection apparatus may be arranged is determined, the drug injection control apparatus 100 may determine, as the drug injection position 510, a point at which the drug is to be discharged when the drug injection apparatus is arranged in the drug injection apparatus arrangement area. The drug injection control apparatus 100 may display, on the medical images 410a, 410b, and 410c, information about the drug injection apparatus arrangement area and information about the drug injection position 510.

When the drug injection position 510 is determined, the drug injection control apparatus 100 determines the electrode insertion positions 520a, 520b, 520c, and 520d where the plurality of electrodes 130 are to be inserted. In FIG. 5, a case in which four electrodes 130 are used is described as an example. Even when the number of electrodes 130 is changed, the electrode insertion positions 520a, 520b, 520c, and 520d may be determined similarly to the case in which the four electrodes 130 are used.

The drug injection control apparatus 100 determines the electrode insertion positions 520a, 520b, 520c, and 520d based on a position and shape of the object of interest 160 and the drug injection position 510. According to an embodiment, the drug injection control apparatus 100 determines a candidate electrode arrangement area around a central area including the object of interest 160 and the drug injection position 510. An area within a certain reference distance from the central area is determined as the candidate electrode arrangement area.

The drug injection control apparatus 100 may determine the electrode insertion positions 520a, 520b, 520c, and 520d in an area of the candidate electrode arrangement area, excluding the access restricted area where access of the electrodes 130 is restricted. As described above, the access restricted area is an area where a tissue or organ with a risk of damage is present in the object 150. The drug injection control apparatus 100 may set the access restricted area based on the position or shape of the object of interest 160.

The drug injection control apparatus 100 determines electrode insertion positions where the four electrodes 130 are arranged, in an area of the candidate electrode arrangement area, excluding the access restricted area. The electrodes 130 have a certain shape and size. The drug injection control apparatus 100 determines the electrode insertion positions in the candidate electrode arrangement area based on the shape and size of the electrodes 130.

The four electrodes 130 have a certain arrangement. According to an embodiment, two of the four electrodes 130 are arranged on both sides in a first-axis direction with the central area therebetween to form a current field that moves the drug 140 in the first-axis direction. Also, the other two of the four electrodes 130 are arranged on both sides in a second-axis direction with the central area therebetween to form a current field that moves the drug 140 in the second-axis direction. A first axis and a second axis may be arranged at a certain angle. According to an embodiment, the first axis and the second axis may be orthogonal to each other. The drug injection control apparatus 100 determines four electrode insertion positions 520a, 520b, 520c, and 520d such that the four electrodes 130 are arranged with the certain arrangement as described above.

According to an embodiment, the drug injection control apparatus 100 determines the drug injection position 510 and the electrode insertion positions 520a, 520b, 520c, and 520d in each of the cross-sectional medical images 410a, 410b, and 410c in the plurality of directions. Also, the drug injection control apparatus 100 displays indicators indicating the drug injection position 510 and the electrode insertion positions 520a, 520b, 520c, and 520d on the medical images 410a, 410b, and 410c and displays the indicators through the output interface 240.

According to an embodiment, the drug injection control apparatus 100 determines the drug injection position 510 and the electrode insertion positions 520a, 520b, 520c, and 520d based on a user input that is input through the input interface 220. According to an embodiment, a user may directly designate the drug injection position 510 and the electrode insertion positions 520a, 520b, 520c, and 520d. For example, the drug injection control apparatus 100 displays information about the object of interest 420 on the medical images 410a, 410b, and 410c and receives a user input for setting the drug injection position 510 and the electrode insertion positions 520a, 520b, 520c, and 520d on the medical images 410a, 410b, and 410c including the information about the object of interest 420. As another example, the drug injection control apparatus 100 displays, on the medical images 410a, 410b, and 410c, information about the object of interest 420, information about the surrounding area of the object of interest 420, and information about the access restricted area and receives a user input for setting the drug injection position 510 on the displayed medical images 410a, 410b, and 410c. As another example, the drug injection control apparatus 100 displays, on the medical images 410a, 410b, and 410c, information about the object of interest 420, information about the drug injection position 510, and information about the central area, and information about the access restricted area and receives a user input for setting the electrode insertion positions 520a, 520b, 520c, and 520d on the displayed medical images 410a, 410b, and 410c.

According to an embodiment, the drug injection control apparatus 100 may adjust, based on a user input, the drug injection position 510 or the electrode insertion positions 520a, 520b, 520c, and 520d, which are automatically set. Also, the drug injection control apparatus 100 may finally determine the drug injection position 510 or the electrode insertion positions 520a, 520b, 520c, and 520d based on a user input that the user finally approves for the automatically set drug injection position 510 or electrode insertion positions 520a, 520b, 520c, and 520d.

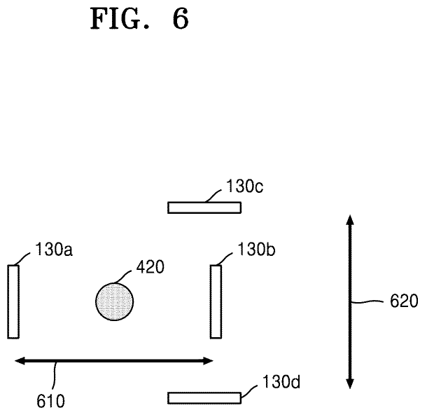

FIG. 6 is a diagram illustrating an arrangement of an object of interest and electrodes, according to an embodiment.

FIG. 6 shows a state in which the object of interest 420 and electrodes 130a, 130b, 130c, and 130d are seen from one direction of the object 150. For example, the object 150 may be a brain, and FIG. 6 may correspond to a horizontal plane of the brain.

According to an embodiment, the four electrodes 130a, 130b, 130c, and 130d are used to move the drug 140 along a desired path over time in a 3D space around the object of interest 420.

The four electrodes 130a, 130b, 130c, and 130d include first-group electrodes 130a and 130b for moving the drug 140 in a first direction 610 and second-group electrodes 130c and 130d for moving the drug 140 in a second direction 620. The first-group electrodes 130a and 130b may be arranged such that an output surface on which a current field is output from the first-group electrodes 130a and 130b is orthogonal to the first direction 610. The second-group electrodes 130c and 130d may be arranged such that an output surface on which a current field is output from the second-group electrodes 130c and 130d is orthogonal to the second direction 620.

The drug injection control apparatus 100 may move the drug 140 in the first direction 610 by applying a current to the first-group electrodes 130a and 130b and generating a current field between the first-group electrodes 130a and 130b. Also, the drug injection control apparatus 100 may move the drug 140 in the second direction 620 by applying a current to the second-group electrodes 130c and 130d and generating a current field between the second-group electrodes 130c and 130d.

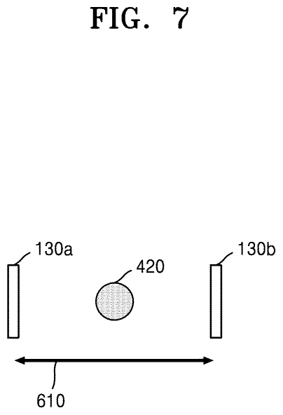

FIG. 7 is a diagram illustrating an arrangement of an object of interest and electrodes, according to an embodiment.

FIG. 7 shows a state in which the object of interest 420 and the electrodes 130a and 130b are seen from one direction of the object 150. For example, the object 150 may be a brain, and FIG. 7 may correspond to a horizontal plane of the brain.

According to an embodiment, the two electrodes 130a and 130b are used to move the drug 140 along a desired path over time in one direction in a 3D space around the object of interest 420.

The two electrodes 130a and 130b move the drug 140 in the first direction 610. The two electrodes 130a and 130b may be arranged such that an output surface on which a current field is output from each of the electrodes 130a and 130b is orthogonal to the first direction 610.

The drug injection control apparatus 100 may move the drug 140 in the first direction 610 by applying a current to the two electrodes 130a and 130b and generating a current field between the two electrodes 130a and 130b.

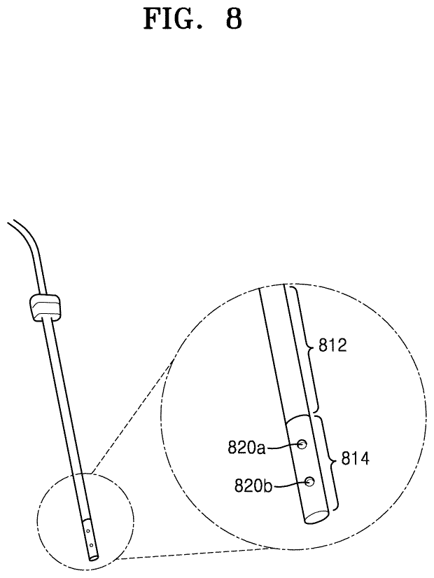

FIG. 8 is a diagram of a drug injection apparatus according to an embodiment.

According to an embodiment, a drug injection apparatus 810 is inserted into the object 150 and injects a drug into the object 150. The drug injection apparatus 810 may have a drug delivery structure and a drug injection force delivery structure. For example, the drug injection apparatus 810 may correspond to a catheter, a needle, or the like.

According to an embodiment, the drug injection apparatus 810 serve as an electrode 130 together. In this case, the drug injection apparatus 810 may correspond to one of the plurality of electrodes 130. For example, the drug injection apparatus 810 corresponds to a catheter. The drug injection apparatus 810 includes an insulating portion 812 and an electrode portion 814. The insulating portion 812 is finished with a material having no or low electrical conductivity. The insulating portion 812 may include, for example, silicon. The electrode portion 814 includes a metal material having electrical conductivity. For example, the electrode portion 814 may include a material such as stainless steel or platinum. The electrode portion 814 may be arranged at a distal end of the drug injection apparatus 810.

The drug injection apparatus 810 may include at least one drug outlet 820a or 820b. The drug injection apparatus 810 discharges the drug 140 into the object 150 through the drug outlets 820a and 820b. According to an embodiment, the drug outlets 820a and 820b may be arranged at the distal end of the drug injection apparatus 810. According to an embodiment, the drug outlets 820a and 820b may be arranged in the electrode portion 814.

The drug injection apparatus 810 may have a size of, for example, 1 mm or greater in diameter.

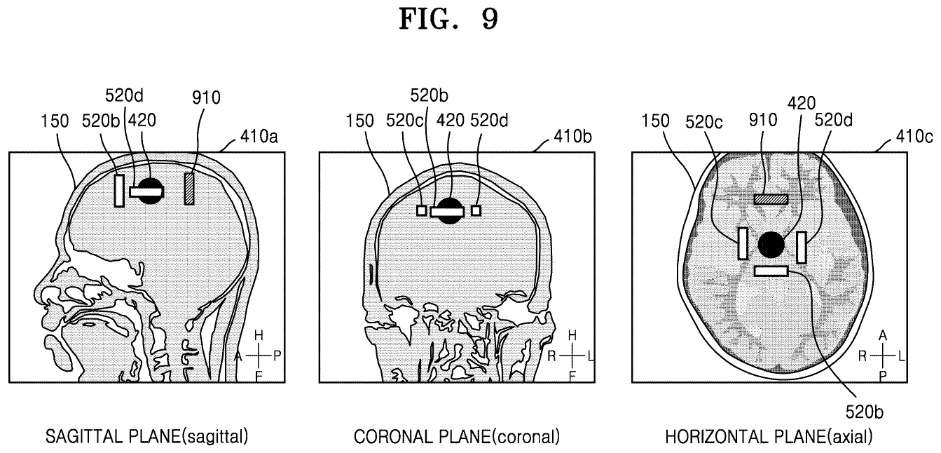

FIG. 9 is a diagram illustrating a drug injection position and a plurality of electrode insertion positions on a medical image, according to an embodiment.

According to an embodiment, the drug injection apparatus 810 includes the electrode portion 814 and serves as the electrode 130. In this case, the drug injection control apparatus 100 sets a drug injection and electrode insertion position 910 where the drug injection apparatus 810 is inserted. The drug injection and electrode insertion position 910 corresponds to a drug injection position and an electrode insertion position. For example, in the embodiment described above with reference to FIG. 5, the drug injection apparatus 810 in the form of a catheter including an electrode is inserted into the electrode insertion position 520a, and the electrode insertion position 520a is set as the drug injection and electrode insertion position 910.

According to an embodiment, the drug injection control apparatus 100 displays indicators indicating the electrode insertion positions 520a, 520b, and 520c and the drug injection and electrode insertion position 910 on the medical images 410a, 410b, and 410c and displays the indicators through the output interface 240. The indicators indicating the electrode insertion positions 520a, 520b, and 520c and the indicator indicating the drug injection and electrode insertion position 910 may have different attributes. For example, the indicators indicating the electrode insertion positions 520a, 520b, and 520c and the indicator indicating the drug injection and electrode insertion position 910 may have different attributes, such as color, pattern, or edge.

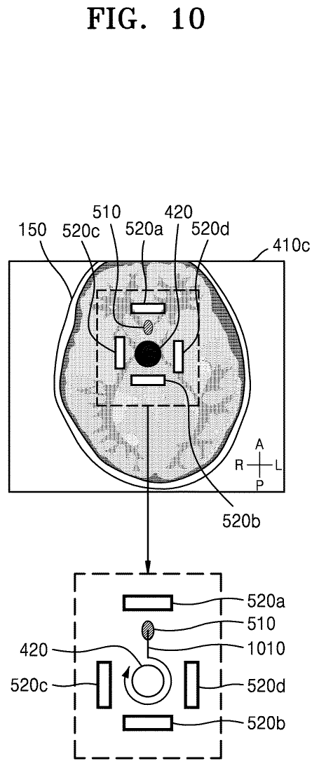

FIG. 10 is a diagram illustrating a process of setting a drug movement path, according to an embodiment.

According to an embodiment, the drug injection control apparatus 100 sets a drug movement path 1010 that is a path along which the drug 140 moves. The drug injection control apparatus 100 sets a path along which the drug 140 moves from the drug injection position 510. The drug movement path 1010 may be set to deliver the drug 140 to the object of interest 420 while the drug 140 moves to the vicinity of the object of interest 420. According to an embodiment, the drug movement path 1010 is set such that the drug 140 moves along the surface of the object of interest 420. The drug injection control apparatus 100 may set the drug movement path 1010 to apply the drug 140 to a part or all of the surface of the object of interest 420.

According to an embodiment, the drug movement path 1010 may be set such that the drug 140 moves to the object of interest 420 and moves along the surface of the object of interest 420.

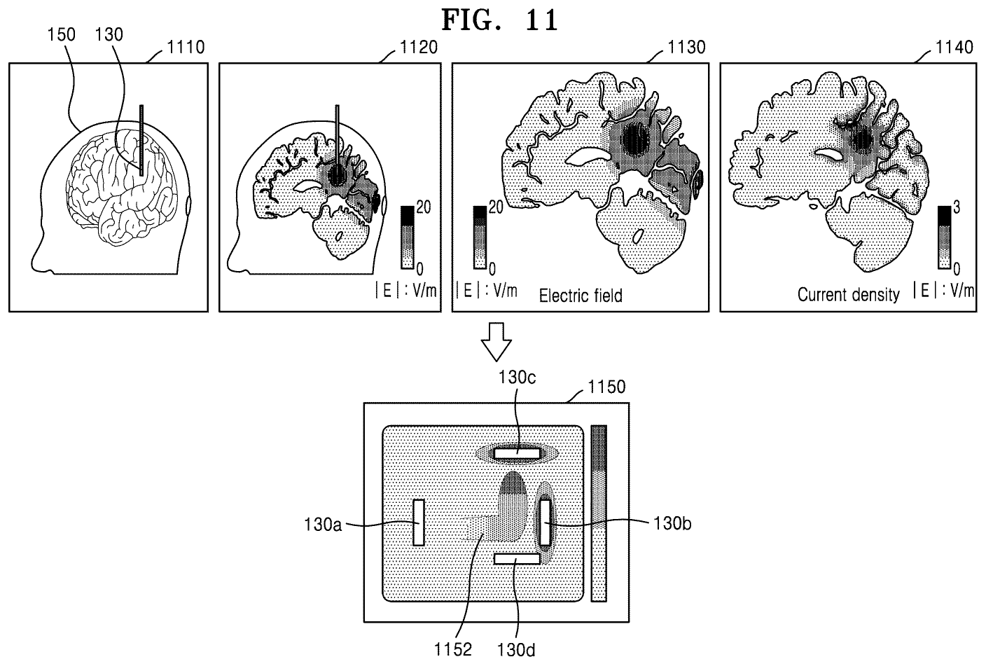

FIG. 11 is a diagram illustrating a process of deriving a current application condition, according to an embodiment.

According to an embodiment, when the electrode 130 is inserted into the object 150 and a current is applied thereto, the drug injection control apparatus 100 derives an optimal current application condition by predicting an electric field and a current field generated in the object 150. In operation 1110, the drug injection control apparatus 100 assumes a situation in which the electrode 130 is arranged at a certain position in the object 150. In operation 1120, the drug injection control apparatus 100 predicts an electric field generated in the object 150 when a current is applied to the electrode 130. The drug injection control apparatus 100 generates an electric field distribution map 1130 and a current density map 1140 based on the predicted electric field. The drug injection control apparatus 100 assumes and simulates the situation in which the electrode 130 is arranged at the certain position as described above, and presents an optimal condition for applying a current.

The drug injection control apparatus 100 may predict an electric field and a current density by assuming a situation in which the electrodes 130 are inserted into a plurality of electrode insertion positions. Also, the drug injection control apparatus 100 may determine an optimal four-dimensional (4D) current application condition by predicting the current density and the electric field generated by a current being output from each electrode 130 and simulating current application conditions over time. For example, in operation 1150, the drug injection control apparatus 100 assumes a situation in which four electrodes 130a, 130b, 130c, and 130d are arranged and predicts an electric field and a current generated in the object 150 when a certain current is applied to the four electrodes 130a, 130b, 130c, and 130d. The drug injection control apparatus 100 determines a current application condition over time to move the drug 140 along the drug movement path 1010 by simulating the current application conditions over time.

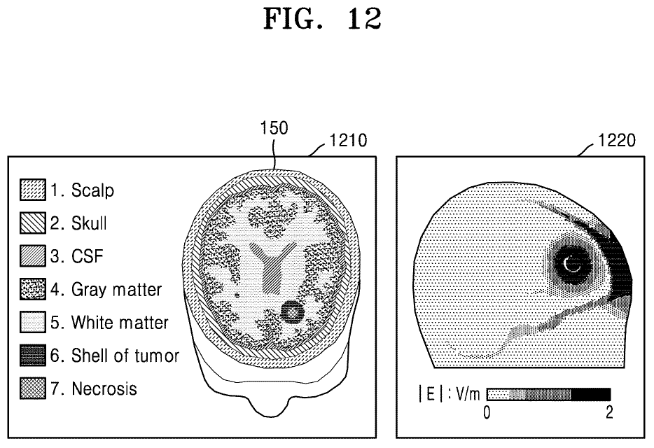

FIG. 12 is a diagram illustrating an example of a current field distribution calculated based on anatomical characteristics of an object, according to an embodiment.

According to an embodiment, the drug injection control apparatus 100 predicts a current field distribution based on anatomical characteristics of the object 150.

The drug injection control apparatus 100 obtains anatomical characteristics 1210 of the object 150. The drug injection control apparatus 100 obtains structural information about each organ or tissue of the object 150 from a medical image. For example, the object 150 is a brain, and the organ or tissue in the object 150 includes scalp, skull, cerebrospinal fluid (CSF), gray matter, white matter, shell of tumor, and necrosis. The anatomical characteristics 1210 may be generated by extracting and integrating pieces of surface data of head components according to a 3D finite element model.

The drug injection control apparatus 100 may generate a current field distribution map 1220 predicted when a current is output from the electrode 130 at a specific position, by using the anatomical characteristics 1210 of the object 150. The drug injection control apparatus 100 may calculate a current field distribution by using the Laplacian equation derived from Maxwell's equation. Each organ or tissue of the object 150 shows different characteristics in the distribution of a current field generated when a current is applied, due to a difference in constituent materials. Because each organ or tissue has a different electrical conductivity, the current field is affected by the anatomical characteristics 1210 of the object 150. The drug injection control apparatus 100 generates the current field distribution map 1220 by individually predicting the distribution of a current field in areas corresponding to each organ and tissue based on the anatomical characteristics 1210 of the object 150.

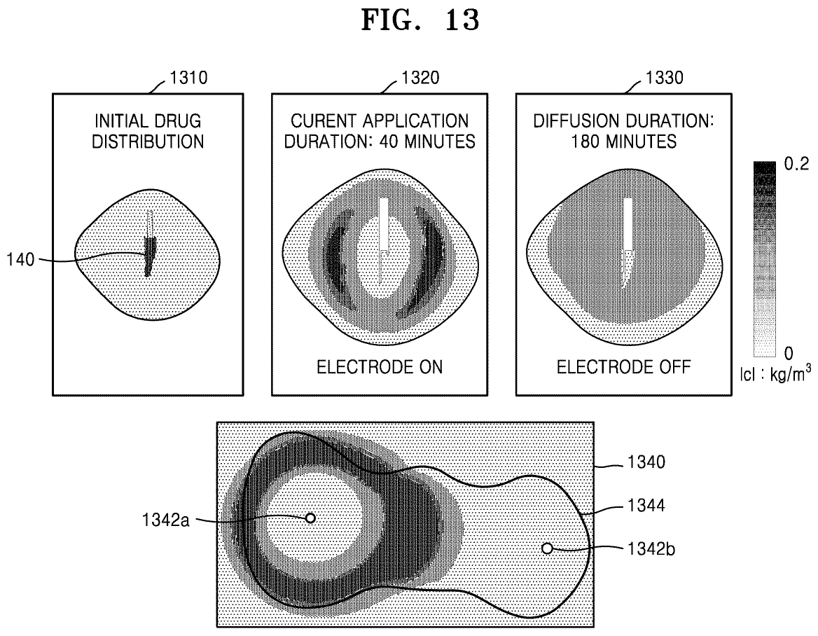

FIG. 13 is a diagram illustrating a state in which a drug is distributed by diffusion and a state in which the drug is distributed over time in a current field, according to an embodiment. In FIG. 13, reference numerals 1310, 1320, 1330, and 1340 represent drug distributions measured in an experiment. A drug corresponds to theranostic ink.

According to an embodiment, when the drug 140 is injected into the object 150 and a current is applied thereto, the drug 140 moves within the object 150 over time. The drug 140 may move according to the electrophoretic principle, iontophoresis principle, or electroosmotic principle within the current field.

In FIG. 13, reference numeral 1310 refers to an initial drug distribution in the object 150, representing a drug distribution immediately after drug injection. The drug 140 is injected into the object 150 at a specific position. After drug injection, a current was output from the electrode 130 arranged at the drug injection position in a first experimental example 1320, and a current was not output from the electrode 130 in a second experimental example 1330. The first experimental example 1320 shows a drug distribution after a current was applied for 40 minutes. The second experimental example 1330 shows a drug distribution when the drug 140 was injected and then passively diffused for 180 minutes without applying a current. When the first experimental example 1320 and the second experimental example 1330 are compared, it may be seen that a drug distribution result of the second experimental example 1330 corresponding to a distribution result of passive diffusion for 180 minutes is similar to a drug distribution result of the first experimental example 1320 corresponding to a result of drug movement within a current field for 40 minutes. Accordingly, it may be seen that moving the drug 140 by the current field, as in the embodiments, is more efficient in view of time compared to the case of passive diffusion.

A third experimental example 1340 shows a result of distributing the drug 140 by generating a current field from two electrodes 1342a and 1342b. The third experimental example 1340 shows a drug distribution when a current is applied for 120 minutes to a long straight brain tumor shape. It may be seen that, when the drug 140 is distributed by generating a current field from the two electrodes 1342a and 1342b inside a boundary 1344 of a tumor, the drug 140 moves in a direction. That is, according to the third experimental example 1340, because a current field in a direction from a first electrode 1342a to a second electrode 1342b is generated, it may be seen that the drug 140 moves in a direction toward the second electrode 1342b.

The drug injection control apparatus 100 may pre-store movement characteristics of the drug 140 according to current intensity, anatomical characteristics, and potential difference. The drug injection control apparatus 100 may predict movement and distribution of the drug 140 over time when a current is output from an electrode at a certain position by using the pre-stored movement characteristics of the drug 140. The drug injection control apparatus 100 may derive an optical current application condition based on a prediction result of the movement and distribution of the drug 140 over time.

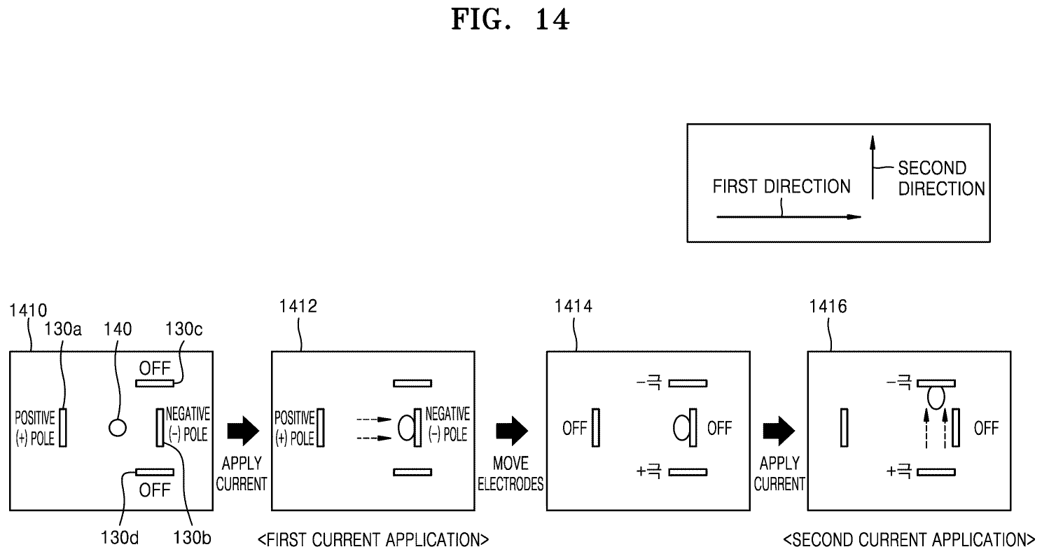

FIG. 14 is a diagram illustrating a state in which a drug is moved by controlling each electrode, according to an embodiment.

According to an embodiment, the drug injection control apparatus 100 controls movement of the drug 140 by turning on/off the plurality of electrodes 130a, 130b, 130c, and 130d. In FIG. 14, an embodiment including four electrodes 130a, 130b, 130c, and 130d is described. The four electrodes 130a, 130b, 130c, and 130d include first-group electrodes 130a and 130b that generate a current in a first direction and second-group electrodes 130c and 130d that generate a current in a second direction.

In FIG. 14, a case in which the drug 140 is moved in the first direction and then moved in the second direction is described. A movement path of the drug 140 may be variously determined according to a set drug movement path.

In order to move the drug 140 in the first direction, the drug injection control apparatus 100 applies a positive voltage to a first electrode 130a and applies a negative voltage to a second electrode 130b in operation 1410. That is, the first electrode 130a operates as a positive (+) pole, and the second electrode 130b operates as a negative (−) pole. A third electrode 130c and a fourth electrode 130d operate in an off state.

Next, in operation 1412, the drug injection control apparatus 100 outputs a current between the first electrode 130a and the second electrode 130b. Accordingly, a first current flowing from the first electrode 130a to the second electrode 130b is generated. The drug 140 moves from the first electrode 130a to the second electrode 130b by the first current flowing between the first electrode 130a and the second electrode 130b.

Next, in operation 1414, the drug injection control apparatus 100 turns off the first electrode 130a and the second electrode 130b and applies a voltage to the third electrode 130c and the fourth electrode 130d. The drug injection control apparatus 100 applies a negative voltage to the third electrode 130c and applies a positive voltage to the fourth electrode 130d. That is, the third electrode 130c operates as a negative (−) pole, and the fourth electrode 130d operates as a positive (+) pole.

Next, in operation 1416, the drug injection control apparatus 100 outputs a current between the third electrode 130c and the fourth electrode 130d. Accordingly, a second current flowing from the fourth electrode 130d to the third electrode 130c is generated. The drug 140 moves from the fourth electrode 130d to the third electrode 130c by the second current flowing between the fourth electrode 130d and the third electrode 130c.

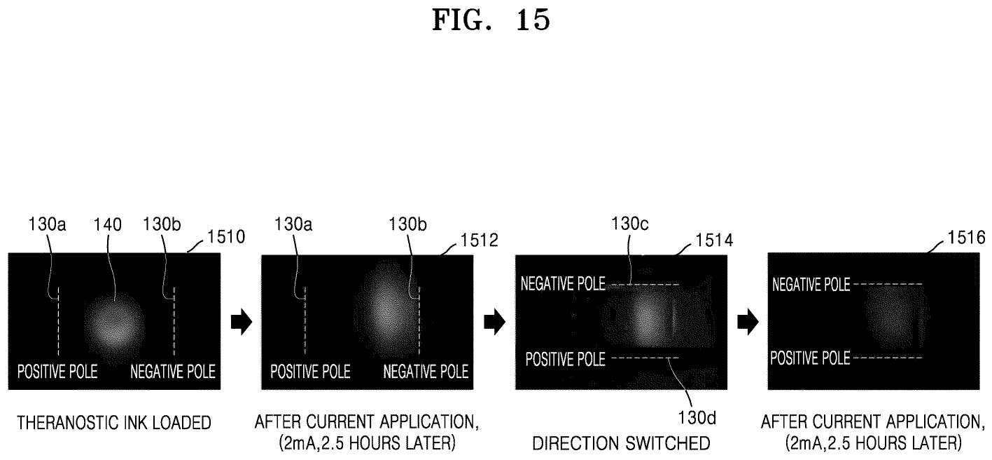

FIG. 15 is an experimental example of moving a drug by applying a current to an electrode, according to an embodiment.

According to an embodiment, movement of the drug 140 was measured after an experiment has been conducted under similar conditions to measure the movement of the drug 140. The drug 140 corresponds to fluorescein (negative charge, molecular weight: 332.31 g/mol). A matrix in which the electrodes 130a, 130b, 130c, and 130d and the drug 140 are injected corresponds to agarose gel (0.5% w/v). A current applied has an intensity of 2 mA. A current application duration is 2.5 hours per direction. For a measurement method, image obtainment using a fluorescence filter used. In the experimental example, molecules were moved by electroosmosis.

In the experimental example of FIG. 15, in operation 1510, a positive (+) voltage was applied to the first electrode 130a, and a negative (−) voltage was applied to the second electrode 130b. The drug 140 was injected between the first electrode 130a and the second electrode 130b.

Next, in operation 1512, a current of 2 mA was applied between the first electrode 130a and the second electrode 130b, and a current application state was maintained for 2.5 hours. In operation 1512, the drug 140 was moved in a direction toward the second electrode 130b corresponding to the negative (−) pole.

Next, in operation 1514, the first electrode 130a and the second electrode 130b were turned off, and the third electrode 130c and the fourth electrode 130d were switched to an on state. A negative (−) voltage was applied to the third electrode 130c, and a positive (+) voltage was applied to the fourth electrode 130d.

Next, in operation 1516, a current of 2 mA was applied between the fourth electrode 130d and the third electrode 130c, and a current application state was maintained for 2.5 hours. In operation 1516, the drug 140 was moved in a direction toward the third electrode 130c corresponding to the negative (−) pole.

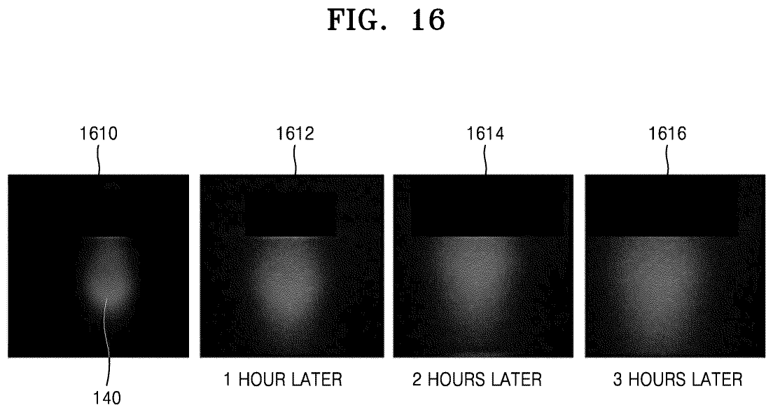

FIG. 16 is an experimental example illustrating a state in which a drug moves in a matrix according to a comparative example.

In a comparative example of FIG. 16, the same matrix and the same drug 140 as the experimental example of FIG. 15 were used. However, in the comparative example of FIG. 16, after only the drug 140 was injected without inserting an electrode, natural diffusion of the drug 140 was observed. For a measurement method, image obtainment using a fluorescence filter is used. In FIG. 16, a state immediately after drug injection 1610, a state of one hour after drug injection 1612, a state of two hours after drug injection 1614, and a state of three hours after drug injection 1616 are shown. For measurement results of FIG. 16, it may be seen that the drug 140 diffuses in a spherical shape without a direction. Also, it may be seen that a movement speed of the drug 140 is lower than that of the experimental example of FIG. 15 in which the current was applied.

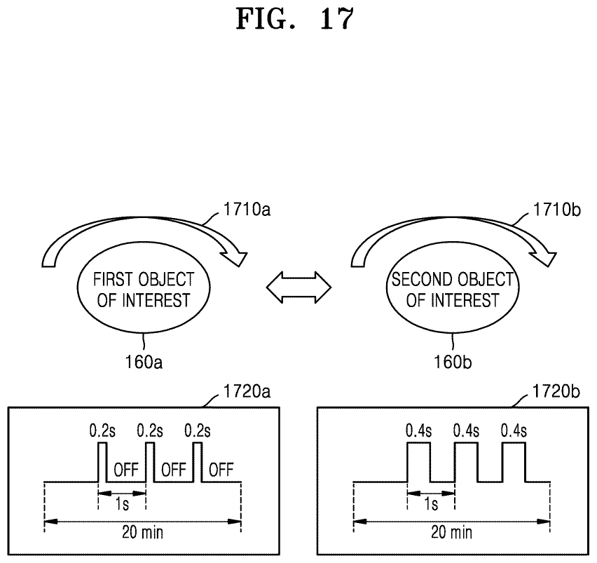

FIG. 17 is a diagram of a configuration for adjusting a movement rate of a drug, according to an embodiment.

According to an embodiment, a movement speed of the drug 140 may be set according to characteristics of objects of interest 160a and 160b. The drug 140 is applied to the surfaces of the objects of interest 160a and 160b and moves and penetrates into the objects of interest 160a and 160b. However, a movement pattern and speed of the drug 140 vary according to the characteristics of the objects of interest 160a and 160b. According to an embodiment, a change in an actual current appearing in a tissue and a change in the movement speed of the drug 140 according to characteristics of objects of interest may be reflected in an output current. For example, because a current flows well through a cancer tissue, the movement speed to the objects of interest 160a and 160b may be faster compared to the case in which the same current is applied to other tissues.

The drug injection control apparatus 100 may adjust the movement speed of the drug 140 by adjusting a duty ratio of a current applied to the electrode 130. The drug injection control apparatus 100 increases the movement speed of the drug 140 by increasing the duty ratio of the current applied to the electrode 130 and reduces the movement speed of the drug 140 by reducing the duty ratio.

According to an embodiment, the drug injection control apparatus 100 may adjust the movement speed of the drug 140 by adjusting the duty ratio of the current applied to the electrode 130 according to the characteristics of the objects of interest. In the example of FIG. 17, a first object of interest 160a has high electrical conductivity, and a second object of interest 160b has low electrical conductivity. The drug injection control apparatus 100 may set a relatively low duty ratio of the current in the first object of interest 160a and set a relatively high duty ratio of a current output from the electrode in the second object of interest 160b. For example, the drug injection control apparatus 100 may determine the duty ratio of the current output from the electrode based on electrical conductivity characteristics of the objects of interest 160a and 160b. The drug injection control apparatus 100 may reduce the duty ratio of the current when the electrical conductivity is high (1720a) and increase the duty ratio of the current when the electrical conductivity is low (1720b). The drug 140 moves along a first path 1710a around the first object of interest 160a or moves along a second path 1710b around the second object of interest 160b according to the duty ratio of the current output from the electrode. The drug injection control apparatus 100 may appropriately adjust the movement speed of the drug 140 in consideration of the electrical conductivity of an object of interest.

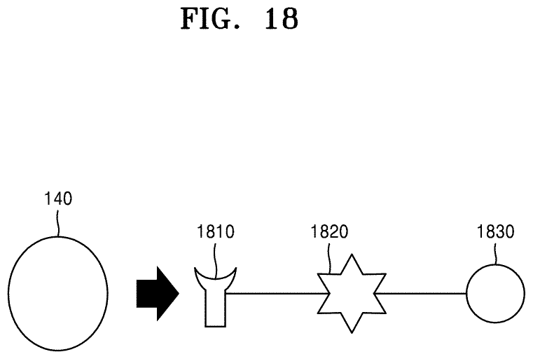

FIG. 18 is a diagram of a structure of a drug according to an embodiment.

According to an embodiment, the drug 140 may correspond to theranostic ink. The theranostic ink may correspond to small molecules or particles.

The theranostic ink may include a tissue-specific adhesion functional group (targeting moiety) 1810, a contrast agent 1820, and an anticancer agent 1830. The theranostic ink has a contrasting function capable of determining a position of distribution in a tissue, selective adhesion to a tissue to be distributed, and an effect of treating lesions.

According to an embodiment, when a current is applied into the object 150, only a current in a range in which tissues are not damaged may be applied. When the object 150 corresponds to a brain, a current of up to 2 mA may be applied to the inside of the brain, and a current of up to 10 mA may be applied to the cerebral cortex. However, in the case of using such a microcurrent, a micro- or nano-sized polymeric drug using the microcurrent is difficult to move. According to an embodiment, the drug 140 corresponds to small molecules or ink and may move even in a low-intensity current field.

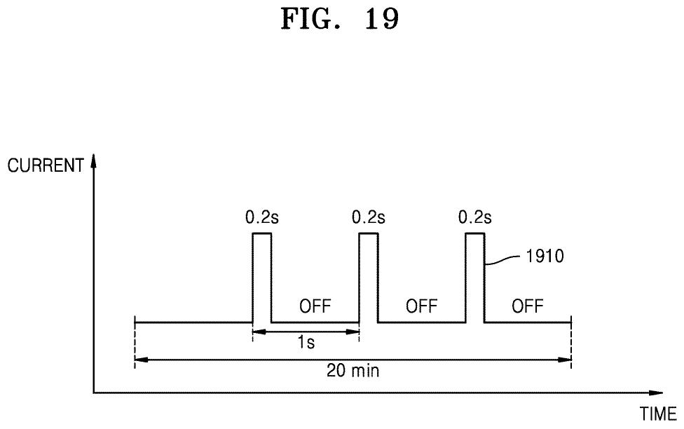

FIG. 19 is a diagram illustrating current sequence information according to an embodiment.

According to an embodiment, the drug injection control apparatus 100 generates current sequence information 1910 that defines a current to be applied over time to each of the plurality of electrodes 130 to move a drug along a drug movement path. The current sequence information 1910 is generated for each of the plurality of electrodes 130. The current sequence information 1910 defines at least one of a current value of a current applied to an electrode, a duty cycle, a total current application duration, or an on/off timing or a combination thereof.

The current value represents the magnitude of the current. The current value may have a positive (+) or negative (−) polarity to indicate a direction of the current. The maximum size of the current value may be defined according to the type of the object 150. For example, when the object 150 is a brain, the maximum current value may be defined as 2 mA. When a patch-type electrode is attached to the outside of the brain, the maximum current value may be defined as 10 mA.

The duty cycle represents a ratio of a time interval in which a current is supplied during a current application cycle. The duty cycle may correspond to a duty ratio. The drug injection control apparatus 100 adjusts the movement speed of the drug 140 by defining the duty cycle. The drug injection control apparatus 100 may increase the duty cycle to accelerate the movement speed of the drug 140. The drug injection control apparatus 100 may reduce the duty cycle to decelerate the movement speed of the drug 140. However, when the duty cycle is high, heat may be generated around the electrode 130 to heat tissues of the object 150. Accordingly, the drug injection control apparatus 100 may set the maximum duty cycle such that the tissues of the object 150 are not heated, and may adjust the duty cycle not to exceed the maximum duty cycle.

The total current application duration is a total duration in which a current is applied. The total current application duration may be determined based on a length of the drug movement path. With respect to the total current application duration, the drug injection control apparatus 100 calculates a high-level interval duration of a current required for the drug 140 to move from a starting point to an end point of the drug movement path, in consideration of the movement speed of the drug 140 when a current field is applied. The drug injection control apparatus 100 calculates the total current application duration to have the high-level interval duration in consideration of the duty cycle. For example, when the duty cycle is 20% and the high-level interval duration is 10 minutes, the total current application duration is set to 50 minutes.

The on/off timing is defined by a waveform of the current sequence information 1910.

The drug injection control apparatus 100 generates the current sequence information 1910 and outputs the current sequence information 1910 to the current source apparatus 110. The current source apparatus 110 generates a current to be applied to the plurality of electrodes 130 based on the current sequence information 1910 and applies the current to the plurality of electrodes 130.

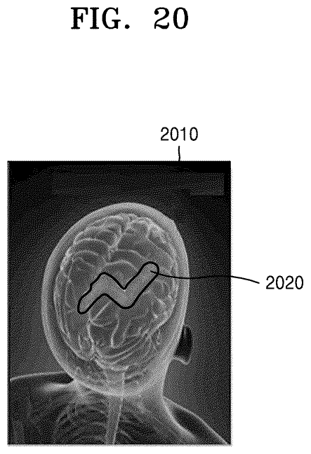

Next, a drug injection control process according to an embodiment will be described with reference to FIGS. 20 to 24.

FIG. 20 is a diagram illustrating a process of identifying an object of interest, according to an embodiment.

According to an embodiment, the drug injection control apparatus 100 identifies an object of interest 2020 from the object 150 based on a medical image 2010. The medical image 2010 may correspond to a 3D medical image. The object of interest 2020 may correspond to a brain tumor.

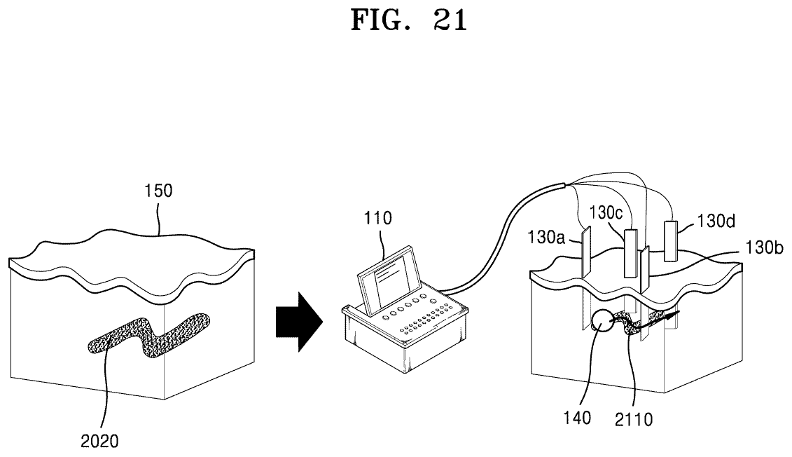

FIG. 21 is a diagram illustrating a process of setting a drug movement path, according to an embodiment.

According to an embodiment, the drug injection control apparatus 100 sets a drug movement path 2110 for applying the drug 140 to the object of interest 2020. The drug injection control apparatus 100 applies a current to the object 150 by using four electrodes 130a, 130b, 130c, and 130d inserted into the object 150. The drug injection control apparatus 100 may precisely move the drug 140 in a 3D space by applying a current to the four electrodes 130a, 130b, 130c, and 130d.

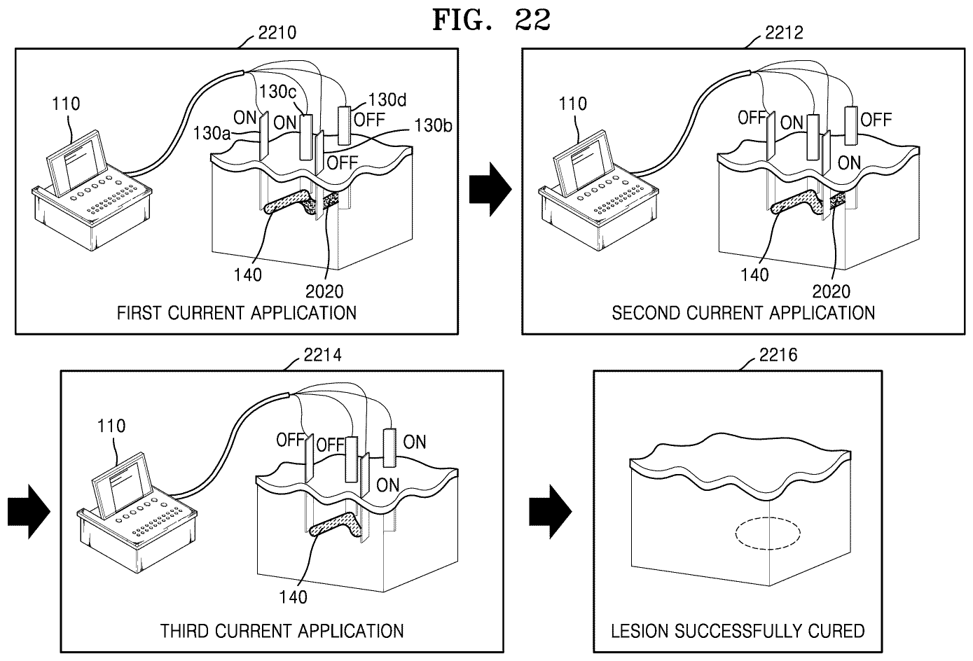

FIG. 22 is a diagram illustrating a state in which a drug is moved by applying a current to four electrodes, according to an embodiment.

According to an embodiment, the drug injection control apparatus 100 applies the drug 140 to the object of interest 2020 by applying a current to the four electrodes 130a, 130b, 130c, and 130d. The drug injection control apparatus 100 may divide the drug movement path 2110 into one or more intervals according to directions, generate currents of different directions, and output the currents through at least some of the four electrodes 130a, 130b, 130c, and 130d. In the embodiment of FIG. 22, three current intervals are set based on a shape of the object of interest 2020.

According to an embodiment, an arrangement of electrodes are determined based on one or more current intervals set based on the drug movement path 2110. For example, two electrodes may be arranged to apply a current in a direction corresponding to a first current interval, and two electrodes may be arranged to apply a current in a direction corresponding to a second current interval. When the directions of the current intervals are on the same axis, currents corresponding to different current intervals may be output using the same electrode.

In a first current application interval 2210, the first electrode 130a and the third electrode 130c are turned on, and a current is applied between the first electrode 130a and the third electrode 130c. In a second current application interval 2212, the second electrode 130b and the third electrode 130c are turned on, and a current is applied between the second electrode 130b and the third electrode 130c. In a third current application interval 2214, the third electrode 130c and the fourth electrode 130d are turned on, and a current is applied between the third electrode 130c and the fourth electrode 130d.

In operation 2216, when the movement of the drug 140 along the drug movement path is completed, the movement of the drug 140 to the object of interest 2020 is completed. The object of interest 2020 may be eliminated or cured by a therapeutic effect of the drug 140.

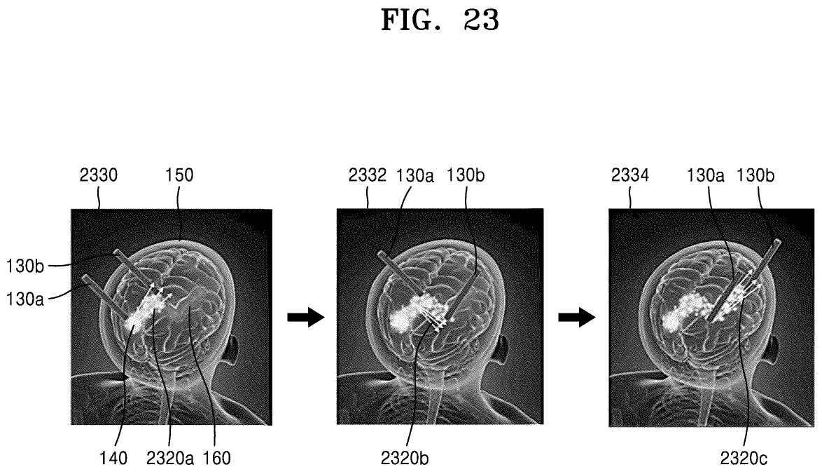

FIG. 23 is a diagram illustrating an arrangement of electrodes and a current application process, according to an embodiment.

According to an embodiment, the delivery process may proceed while changing insertion positions of the electrodes 130a and 130b during the drug delivery process. The drug injection control apparatus 100 determines an electrode insertion position that is sequentially moved, based on a position and shape of the object of interest 160. An electrode insertion process and a drug delivery process be performed through a plurality of operations. For example, as shown in FIG. 23, two electrodes 130a and 130b are inserted into the object 150, and the two electrodes 130a and 130b are inserted into the object 150 three times. Accordingly, the electrode insertion process is performed three times.

First, in operation 2330, the first electrode 130a is inserted into an eleventh position, and the second electrode 130b is inserted into a twelfth position. When the first electrode 130a and the second electrode 130b are inserted into the object 150, a current is output to the first electrode 130a and the second electrode 130b, a current field 2320a is generated between the first electrode 130a and the second electrode 130b, and the drug 140 is moved by the current field 2320a. After the current is output to the first electrode 130a and the second electrode 130b for a set time, the first electrode 130a and the second electrode 130b are turned off. Next, the first electrode 130a and the second electrode 130b are removed from the object 150.

Next, in operation 2332, the first electrode 130a is inserted into the twelfth position, and the second electrode 130b is inserted into a twenty-second position. When the first electrode 130a and the second electrode 130b are inserted into the object 150, the current is output to the first electrode 130a and the second electrode 130b, a current field 2320b is generated between the first electrode 130a and the second electrode 130b, and the drug 140 is moved by the current field 2320b. After the current is output to the first electrode 130a and the second electrode 130b for a set time, the first electrode 130a and the second electrode 130b are turned off. Next, the first electrode 130a and the second electrode 130b are removed from the object 150.

Next, in operation 2334, the first electrode 130a is inserted into a thirteenth position, and the second electrode 130b is inserted into a twenty-third position. When the first electrode 130a and the second electrode 130b are inserted into the object 150, the current is output to the first electrode 130a and the second electrode 130b, a current field 2320c is generated between the first electrode 130a and the second electrode 130b, and the drug 140 is moved by the current field 2320c. After the current is output to the first electrode 130a and the second electrode 130b for a set time, the first electrode 130a and the second electrode 130b are turned off. Next, the first electrode 130a and the second electrode 130b are removed from the object 150.

The drug injection control apparatus 100 determines whether there is need to insert the electrodes 130a and 130b through a plurality of operations based on a medical image. For example, the drug injection control apparatus 100 may determine to insert the electrodes 130a and 130b twice or more when the drug 140 is difficult to be delivered to the object of interest 160 by using the electrodes 130a and 130b at fixed positions, when the number of the electrodes 130a and 130b is limited, when the number of the electrodes 130a and 130b is limited by a structure of the object 150 or the object of interest 160, and the like. In this case, the drug injection control apparatus 100 determines positions for a plurality of times of electrode insertion and determines a drug movement path and current sequence information at each electrode insertion position. Also, the drug injection control apparatus 100 outputs, through each output interface 240, information about the positions for the plurality of times of electrode insertion and information about a current application duration at each electrode insertion position. While the drug delivery process is in progress, the drug injection control apparatus 100 may output, through the output interface 240, progress information about a process in which electrode insertion and current application are performed. Also, after the electrode insertion is completed, the drug injection control apparatus 100 outputs information about a current application duration at a corresponding electrode insertion position and outputs information about the time that has elapsed since the current application. Also, when the current application duration at the corresponding electrode insertion position is completed, the drug injection control apparatus 100 outputs information indicating that the current application has been completed.

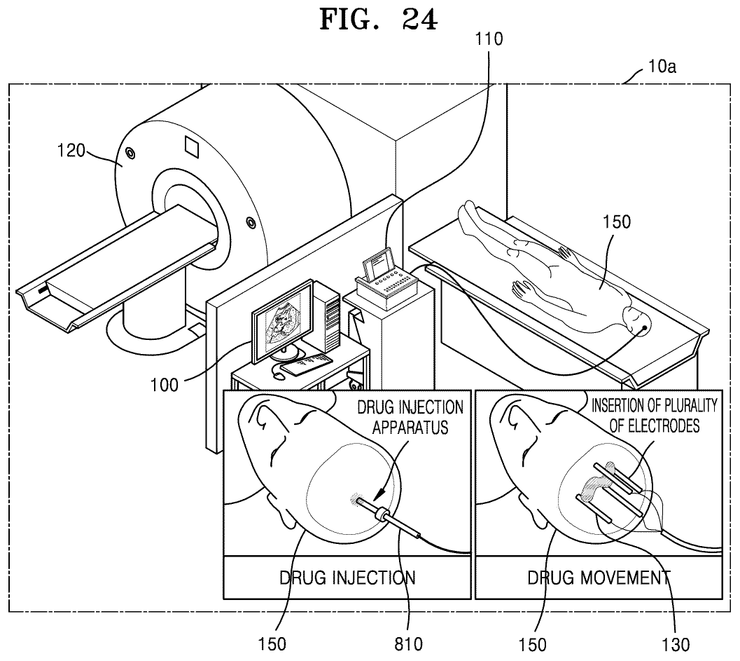

FIG. 24 is a diagram of a drug injection control system 10a according to an embodiment.

The drug injection control system 10a captures a medical image by using a medical imaging apparatus 120. The drug injection control apparatus 100 receives a medical image of the object 150 from the medical imaging apparatus 120. The drug injection control apparatus 100 executes 4D drug movement simulation software that identifies an object of interest within the object 150 and controls movement of a drug. The drug injection control apparatus 100 identifies the object of interest according to a result of the drug movement simulation. Also, the drug injection control apparatus 100 determines a drug injection position and electrode insertion positions according to the result of the drug movement simulation. A drug is injected into the determined drug injection position by using the drug injection apparatus 810. Also, the electrodes 130 are inserted into the determined electrode insertion positions. The drug injection control apparatus 100 determines a drug movement path and generates current sequence information to be applied to each electrode 130. The drug injection control apparatus 100 outputs the current sequence information to the current source apparatus 110. The current source apparatus 110 generates a current based on the current sequence information and outputs the current to the plurality of electrodes 130 inserted into the object 150. The drug is moved by the current output to the plurality of electrodes 130 and is delivered to the object of interest.

As used in various embodiments set forth herein, the term “module” may include a unit implemented in hardware, software, or firmware, and may interchangeably be used with other terms, for example, logic, logic block, part, or circuitry. The module may be an integrated component or a part or a minimum unit of the integrated component that performs one or more functions. For example, according to an embodiment, the module may be implemented in a form of an application-specific integrated circuit (ASIC).

Various embodiments as set forth herein may be implemented as software (e.g., a program) including one or more instructions that are stored in a storage medium that is readable by a machine (e.g., the drug injection control apparatus 100). For example, a processor of the machine (e.g., the drug injection control apparatus 100) may invoke at least one of the one or more instructions stored in the storage medium and execute the same. This allows the machine to be operated to perform at least one function according to the at least one instruction invoked. The one or more instructions may include code generated by a complier or code executable by an interpreter. A machine-readable storage medium may be provided in a form of a non-transitory storage medium. The term “non-transitory” simply means that the storage medium is a tangible apparatus and does not include a signal (e.g., an electromagnetic wave), but this term does not differentiate between where data is semi-permanently stored in the storage medium and where the data is temporarily stored in the storage medium.

According to an embodiment, a method according to various embodiments set forth herein may be provided by being included in a computer program product. The computer program product may be traded as a product between a seller and a buyer. The computer program product may be distributed in a form of machine-readable storage medium (e.g., a compact disc (CD)-ROM)), or distributed (e.g., downloaded or uploaded) through an application store or directly or online between two user apparatuses (e.g., smartphones). When distributed online, at least a part of the computer program product may be temporarily generated or at least temporarily stored in the machine-readable storage medium, such as a memory of a manufacturer's server, a server of an application store, or a relay server.

According to various embodiments, each of the aforementioned components (e.g., modules or programs) may include a single or a plurality of entities, and some of the plurality of entities may be separately arranged in another component. According to various embodiments, one or more of the aforementioned components or operations may be omitted, or one or more other components or operations may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, one or more of the operations may be executed in a different order or omitted, or one or more other operations may be added.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the disclosure as defined by the following claims.

What is claimed is:

1. A method of controlling injection of a drug, the method comprising:
receiving a captured medical image of an object;
identifying an object of interest from the medical image;
based on a position and a shape of the object of interest, determining a drug injection position where a drug is to be injected;
based on the position and the shape of the object of interest, determining a plurality of electrode insertion positions where a plurality of electrodes are to be inserted, respectively, the plurality of electrodes being configured to supply a current to move the drug;
defining a drug movement path based on a shape of the identified object of interest;
generating current sequence information to move the drug along the drug movement path, the current sequence information defining a current to be applied over time to each of the plurality of electrode; and
outputting the current sequence information to a current source apparatus, the current source apparatus being configured to supply the current to the plurality of electrodes.

2. The method of claim 1, wherein the defining of the drug movement path comprises defining the drug movement path to move the drug along a surface of the object of interest or defining the drug movement path to move the drug into the object of interest.

3. The method of claim 1, wherein the object is a brain, and
a magnitude of the current supplied to the plurality of electrodes is defined as a 2 mA or less.

4. The method of claim 1, wherein the current sequence information comprises at least one of a current value of the current applied to each of the plurality of electrodes, a duty cycle, a total current application duration, and an on/off timing or a combination thereof.

5. The method of claim 4, wherein the generating of the current sequence information comprises determining the total current application duration based on a movement distance for which the drug moves along the drug movement path and the duty cycle.

6. The method of claim 4, wherein the generating of the current sequence information further comprises determining an on/off timing of each of the plurality of electrodes to move the drug along the drug movement path.

7. The method of claim 4, wherein the generating of the current sequence information further comprises:
determining a movement speed of the drug based on the shape of the object of interest; and
determining the duty cycle based on the movement speed of the drug.

8. The method of claim 7, wherein the determining of the movement speed of the drug comprises increasing the movement speed of the drug as electrical conductivity of the object of interest is lower.

9. The method of claim 1, wherein the injection of the drug is performed by inserting a drug injection apparatus at the drug injection position in the object and injecting the drug from the drug injection apparatus into the object, and
when the drug injection apparatus comprises an electrode, the drug injection position corresponds to one of the plurality of electrode insertion positions.

10. The method of claim 1, wherein the object of interest corresponds to a lesion or a tumor in the object.

11. The method of claim 1, wherein the medical image comprises at least one of a magnetic resonance imaging (MRI) image, a computed tomography (CT) image, an X-ray image, or an ultrasound image.

12. The method of claim 1, wherein the drug comprises at least one of polar molecules, ions, or particles.

13. The method of claim 1, wherein the drug comprises at least one of a tissue-specific adhesion functional group for the object of interest, a contrast agent, or a therapeutic agent.

14. The method of claim 1, further comprising displaying, on the medical image, object-of-interest region information corresponding to the object of interest, information about the drug injection position, information about the plurality of electrode insertion positions, and information about the drug movement path.

15. An apparatus for controlling injection of a drug, the apparatus comprising:
an input interface;
an output interface;
a memory storing at least one instruction; and
at least one processor connected to the memory,
wherein the at least one processor is configured to execute the at least one instruction to:
receive a captured medical image of an object through the input interface;
identify an object of interest from the medical image;
based on a position and a shape of the object of interest, determine a drug injection position where a drug is to be injected;
based on the position and the shape of the object of interest, determine a plurality of electrode insertion positions where a plurality of electrodes are to be inserted, the plurality of electrodes being configured to supply a current to move the drug;
define a drug movement path defined based on a shape of the identified object of interest;
generate current sequence information to move the drug along the drug movement path, the current sequence information defining a current applied over time to each of the plurality of electrodes; and
output the current sequence information to a current source apparatus through the output interface, the current source apparatus being configured to supply the current to the plurality of electrodes.

16. The apparatus of claim 15, wherein the at least one processor is further configured to execute the at least one instruction to define the drug movement path to move the drug along a surface of the object of interest or define the drug movement path to move the drug into the object of interest.

17. The apparatus of claim 15, wherein the object is a brain, and
a magnitude of the current applied to the plurality of electrodes is defined as 2 mA or less.

18. The apparatus of claim 15, wherein the current sequence information comprises at least one of a current value of the current applied to each of the plurality of electrodes, a duty cycle, a total current application duration, and an on/off timing or a combination thereof.

19. The apparatus of claim 18, wherein the at least one processor is further configured to execute the at least one instruction to determine the total current application duration based on a movement distance for which the drug moves along the drug movement path and the duty cycle.

20. A computer-readable recording medium having recorded thereon a program for executing, on a computer, the method of claim 1.
